(19) United States
(12) Patent Application Publication
(10) Pub. No.: US 2012/0220944 A1
(43) Pub. Date: Aug. 30, 2012

(54) CANNULA DEVICE

(75) Inventor: Jarl Charlez, Askim (SE)

(73) Assignee: Lavin AB, Vaxholm (SE)

(21) Appl. No.: 13/510,287

(22) PCT Filed: Nov. 17, 2010
(86) PCT No.: PCT/EP2010/067699

§ 371 Date: May 16, 2012

(30) Foreign Application Priority Data
Nov. 18, 2009 (SE) 0901464.8

(51) Int. Cl. A61M 25/02; A61B 17/34

(52) U.S. Cl. 604/164.04; 604/174

(57) ABSTRACT

A vascular access device (100) suitable for evacuating air from a blood vessel having a vessel wall comprising a cannula (105) for insertion into the blood vessel; wherein an anchoring organ (101) having a first end (102) and a second end (112), the first end (102) of the anchoring organ being fixed to a first end (103) of the cannula (105), and the anchoring organ (101) being adapted to alter its shape between an out-spread shape and a streamlined shape upon a movement being applied to it;-a linkage (119, 120, 131, 132, 141, 142) comprising:-a first sleeve portion (119) being slideable along the cannula (105);-a second sleeve portion (120) being fixedly attached to the cannula (105);-a first outer arm (131) and a second outer arm (132), symetrically arranged relatively to the cannula (105), and opposite each other, the first outer arm (131) and the second outer arm (132) being mechanically connected to the first sleeve portion (119) and being pressable by a persons fingers, wherein the length of the arms is adapted to transfer a movement from the pressing to a movement stretching the anchoring organ (101).

### TECHNICAL FIELD

The present invention is in the field of medical technology and relates to a vascular access device suitable for use during cardiac surgical procedures and particularly it relates to a cannula device for evacuating air from the aorta root before weaning from a heart lung machine. The cannula may also be used to infuse physiological or medical solutions.

### BACKGROUND ART

During various cardiac medical procedures it is desirable to place a cannula within a patient's blood vessel for the purpose of evacuating air from the inside of the vessel in order to prevent air embolism. There are some standard aortic root cannulas on the market today that can be used to penetrate the aortic vessel wall to create a passage for trapped air out of the aorta, but while these standard devices and their associated techniques are extremely useful during certain procedures, they also have significant limitations. These types of standard devices need a certain suture technique for fixation to the vessel and they tend to move about after insertion, which may result in dislodgement from the desired position. Such movement of the cannula may lead to the catheter tip injuring the blood vessel wall.

U.S. Pat. No. 5,531,935 disclose a cannula assembly for aspirating air from the aorta, also useable for delivering cardioplegic fluid to the same aorta. The assembly includes a hub having a single needle portion. The needle portion has an inner cannula which extends through the hub and defines an inner lumen through which cardioplegic fluid may be injected. An outer cannula is coaxially positioned over the inner cannula and is diametrically spaced therefrom to define an outer lumen. A plurality of holes is formed in the sides of the outer cannula through which air can be aspirated.

### SUMMARY OF THE INVENTION

The inventors have also identified that a problem with prior art devices may be that the cannula may be tilted or dislocated such that it becomes occluded by interaction with the blood vessel wall.

The objective of the present invention is to provide a device that overcomes some of the drawbacks mentioned above. A particular objective is to provide a device for evacuating air trapped in the aorta and which can be attached to the aorta wall without the need of one or more sutures, which can be fast and easily attached, and which do not move about or tilt after insertion. It is a further objective to provide a device which does not dislodge from a desired position, and which does not injure the blood vessel wall.

Therefore, the present invention provides a cannula device for vascular access that can be advantageously used during cardiac surgical procedures and particularly for evacuating air from the aorta root before weaning from a heart lung machine. The device incorporates means for air evacuation, means for air detection, and an anchoring system for attaching the device to the blood vessel by clamping rather than attaching the device to the blood vessel by suture(s).

According to a first aspect of the invention there is provided a vascular access device suitable for evacuating air from a blood vessel having a vessel wall comprising a cannula for insertion into the blood vessel, and comprising an anchoring organ having a first end and a second end, the first end of the anchoring organ being fixed to a first end of the cannula, and the anchoring organ being adapted to alter its shape between an out-spread shape and a streamlined shape upon a movement being applied to it. Further the device comprising a linkage having a first sleeve portion being slideable along the cannula and a second sleeve portion being fixedly attached to the cannula and a first outer arm and a second outer arm, symmetrically arranged relatively to the cannula, and opposite each other, the first outer arm and the second outer arm being mechanically connected to the first sleeve portion and being pressable by a persons fingers, wherein the length of the arms is adapted to transfer a movement from the pressing to a movement stretching the anchoring organ.

The anchoring organ being arranged to assume the streamlined shape upon pressing the outer arms, enabling the anchoring organ to be inserted through an opening in the wall of the blood vessel, and wherein the resilience of the hinges of the linkage are adapted to allow the anchoring organ to resume the first, out-spread shape, when the pressure is removed, thereby clamping the blood vessel wall between an outspread portion of the anchoring organ and the first sleeve portion of the linkage.

The device wherein the cannula is arranged to transfer a fluid from the blood vessel to a receptacle fixed to the second sleeve portion, the fluid may be a blood-air mixture, and wherein the air portion is supposed to decrease down to zero percent during an air evacuation procedure, and wherein the receptacle is arranged to enable a viewer to observe air bubbles forming in the outpouring fluid.

The device may be provided with first connecting means to connect the receptacle to an active suction device. The device may be provided with second connecting means to connect the receptacle to an infusion aggregate. The first and second connecting means may be identical or the same.

The device may be provided with a puncture needle for puncturing the blood vessel, being concentrically arranged inside the cannula and attached to a knob. The knob may be arranged to be able to press with a person's index finger when holding the device between the persons thumb and middle finger, all three fingers of the same hand.

The knob may be spring loaded to retract the puncture needle to a position wherein the tip is protected by the cannula, when the knob is not pressed.

The device may provided with a puncture needle concentrically arranged inside the cannula and being connected to a cap, the cap being connected to the outer arms via cap arms such that a movement pressing together the outer arms is transferred to a deployment of the puncture needle. Here, the device may further comprise a mechanism for deploying and retracting the puncture needle in a cyclic pattern wherein the deployed state is less frequent than the retracted state upon repeated pressing of the outer arms.

### BRIEF DESCRIPTION OF THE DRAWINGS

The invention will be explained in the following by way of example embodiments and the accompanying drawings of which

FIG. 1a is an oblique view of a cannula device.

FIG. 1b is a front view of the cannula device of FIG. 1a.

FIG. 1c is a side view of the cannula device of FIG. 1a.

FIG. 1d shows a cross sectional view as marked in FIG. 1c.

FIG. 2a is a front view of a cannula device with a grooved cap for needle control.

FIG. 2b is a front view of the cannula of FIG. 2a where an anchoring organ is in a streamlined position and the needle is in a protrusive position.

FIG. 3a shows the various parts, some also in cross section, of a protrusion-retraction mechanism for a puncture needle of the cannula device of FIG. 2a.

FIG. 3b is a fold-open view of a grooved cap of the protrusion-retraction mechanism of FIG. 3a.

FIG. 4a is an oblique view of a cannula device with connector for suction/infusion FIG. 4b is a cross sectional view of the cannula device of FIG. 4a.

### DETAILED DESCRIPTION

### Definitions

“Out-spread shape”, the term “out-spread shape” is in this document used to denote that a shape of a deformable object is in a state of large diameter, and short length i.e., of expanded diameter, in contrast to a state of a nominal or reduced diameter.

“Streamlined shape”, the term “streamlined shape” is in this document used to denote that a shape of a deformable object is in a state of small diameter, i.e., of reduced diameter, in contrast to a state of a nominal or expanded diameter. The reduced diameter usually goes with an expanded length. The streamlined shape allows easier introduction of the object through a narrow passage, aperture, opening, or the like.

### Cannula Device

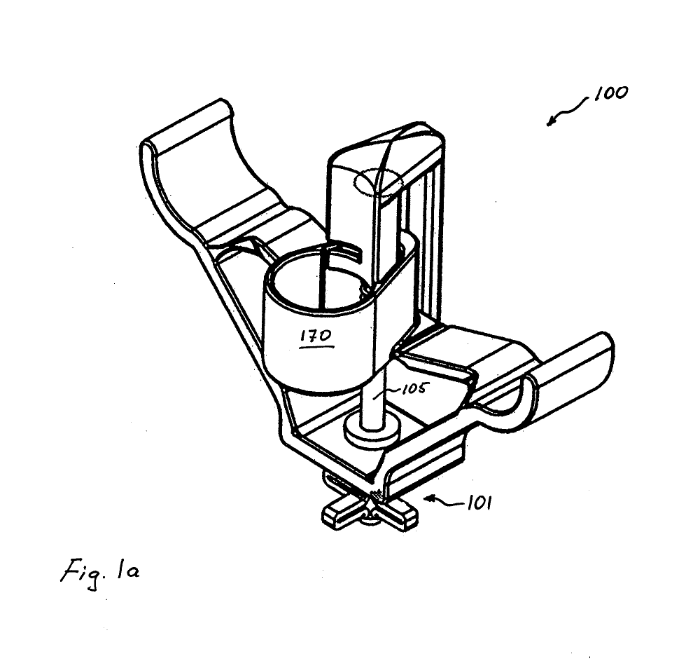

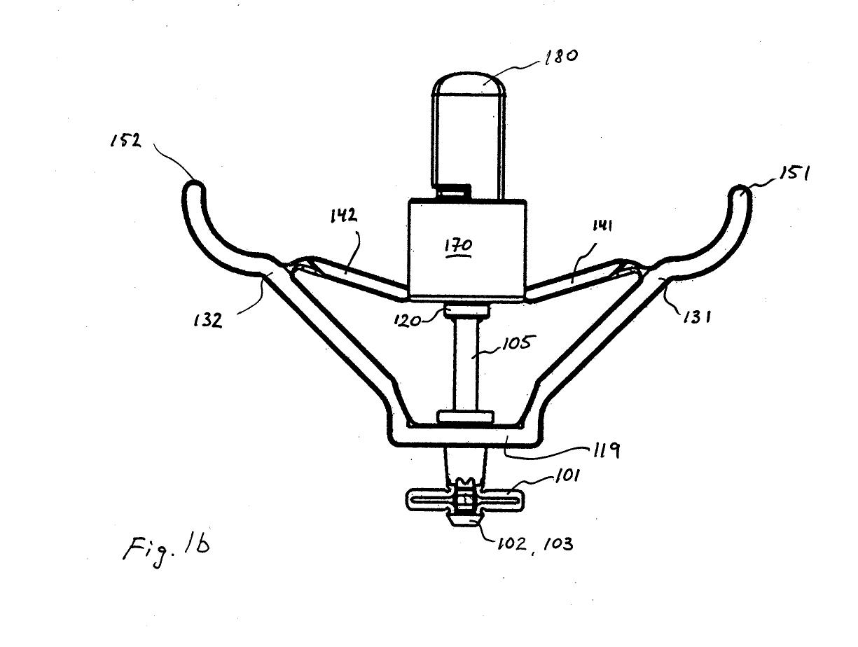

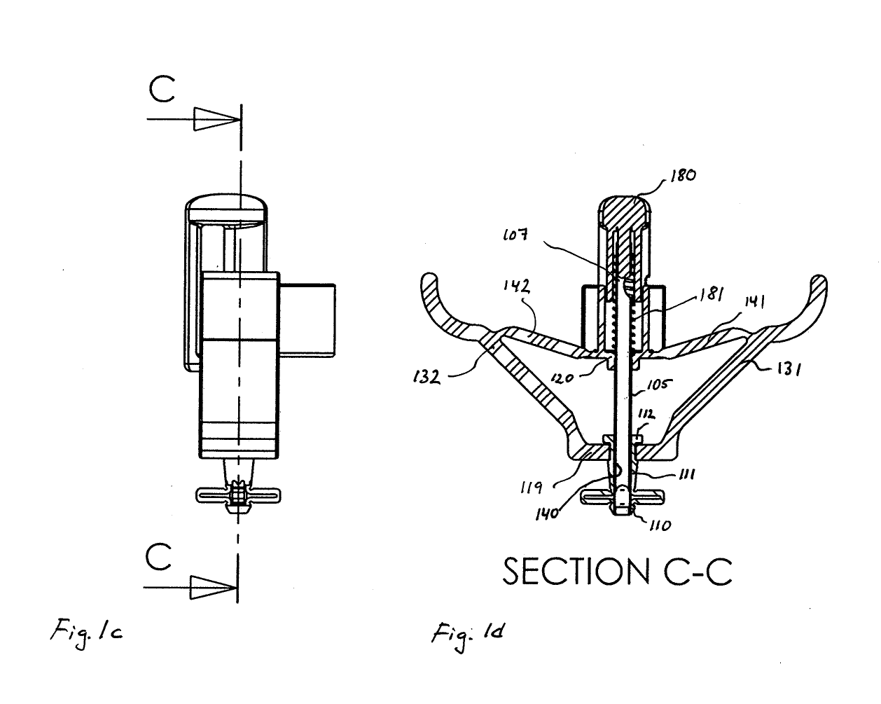

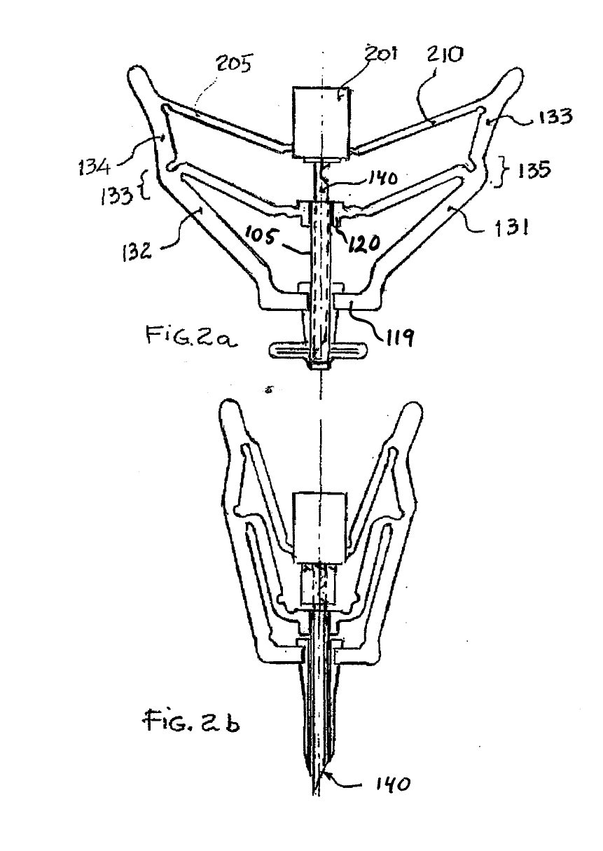

FIG. 1a, 1b, 1c, and 1d shows a cannula device 100 useable for penetrating the aorta wall and for venting air from the aorta during cardiac surgery. The device 100 comprises a cannula 105 for conducting fluid from the blood vessel and out, having a first end 103 to be placed in the blood vessel, and a second end 107 opening out at a small receptacle 170.

### Anchoring Organ

The cannula device further comprises an anchoring organ 101 for attaching the device to the blood vessel. The anchoring organ 101 is made of a material, and are given a shape, that allows it to assume a considerably more streamlined shape when subjected to tensile stress compared to when subjected to mild compression strain. The anchoring organ 101 may preferably be a short slitted tube of a resilient polymer material. Typical dimensions may be: inner diameter 1.5-1.8 mm, outer diameter 1.8-2.2 mm, and length 10-17 mm. Typically, slits may be arranged approximately 1.5-2.0 mm from the first end 102 of the anchoring organ 101 and extending 4.0-6.0 mm in a direction towards a second end 112 of the anchoring organ 101. Typically four slits would be arranged evenly positioned around the circumference of such a rubber tube of the anchoring organ 101. The first end 102 of the anchoring organ 101 is fixed to the first end 103 of the cannula 105. The second end 112 of the anchoring organ 101 is fixed to a first sleeve portion 119 of a linkage, see below, letting the cannula 105 pass concentrically through the anchoring organ 101.

### Linkage And Attachment Sites

Further the device comprises a linkage, i.e., one or more parts connected by hinges or areas of material of reduced thickness, working like hinges. The linkage is adapted to transfer force from a finger pressure on surfaces of the linkage to the anchoring organ 101. The linkage comprises a first sleeve portion 119 having a through-hole adapted to let the cannula slide through it. The linkage further comprises a second sleeve portion 120 to which the cannula 105 is fixed. The first sleeve portion 119 of the linkage is arranged closer to the first end portion 103 of the cannula 105. A first end of a left inner arm 142 is connected to the second sleeve portion 120. A second end of the inner left arm is connected to an outer left arm 132 at a point a first distance from a second end 152 of the outer left arm 132. This first distance may be zero. A first end of the outer left arm is connected to the first sleeve portion 119. The inner left arm is made shorter than the outer left arm. The arms are arranged such that, upon pressure by a persons finger, the first sleeve portion slides relatively to the cannula 105 and drags with it the second end 112 of the anchoring organ, thereby making the anchoring organ 101 assume a streamlined shape. The arms and the hinges are given such a resilience that when not subjected to pressure, i.e., at a rest position, the outer arms form an angle between each other of about 60 degrees thereby forcing the first sleeve portion 119 closer to the cannula tip 102 thereby making the anchoring organ 101 to assume the outspread shape. At an activated (pressed) position the left and the right outer arms are adapted to be parallel or close to parallel.

Thus, the cannula device of the present invention comprises a cannula 105 in the form of a short tube 105 of a rigid material e.g. a metal, for creating a passage through the blood vessel wall. As already mentioned above, the cannula device further comprises an anchoring organ 101 fixed to the short tube of a cannula 105. The anchoring organ 101 has a first and a second end, the first end 102 of the anchoring organ 101 being fixed to a first end 103 of the cannula 105. The anchoring organ 101 is designed to alter its shape between an out-spread shape and a streamlined shape upon a movement being applied to its second end. In FIG. 1a and 1b the anchoring organ is shown in the spread-out shape. Upon pressing outer arms 131 and 132 together the anchoring organ 101 is made to assume the streamlined shape as described above. The anchoring organ 101 is thus arranged to be able to quickly and safely be inserted through the aortic wall into the lumen of the aorta, and is designed for anchoring the device to the wall of the aorta. For features and procedure of punctuating the blood vessel, see the section on “puncture needle” below.

A pair of arms 131, 132 is thus arranged with a first end attached to the anchoring organ 101. A second end is via a linkage connected to the cannula 105. A movement is transferred from the arms to the anchoring element which temporarily alters the shape of the anchoring element into a streamlined configuration, enabling it to easily penetrate through the wall of the vessel. When the arms of the device are released, the anchoring element regain its original shape, i.e., it expands in one or more direction(s) perpendicular to the direction of penetration, thereby engaging the inside wall of the blood vessel.

### Puncture Needle

A puncture needle is preferably arranged concentrically inside the tube 105, which makes it possible to puncture the aorta upon insertion by pressing a knob 180. The puncture needle is preferably arranged to retract automatically by the aid of a spring 181 abutting the second sleeve 120, not to risk damaging blood vessel tissue. The cannula device is thus devised to be held at the outer arms 131, 132 between the thumb and the middle finger, with the index finger free to manoeuvre the puncture needle via the knob 180.

As an alternative, it is also possible to connect the puncture needle to the outer arms 131, 132 such that the puncture needle is only deployed upon insertion, and is then refracted automatically, so as not to be deployed again when removing the device. An alternative is to have a puncture needle which is removed manually after insertion.

After the surgical procedure in question has been terminated the device is easily removed by pressing the legs of the device together and thereby stretching the anchoring organ into the streamlined conformation which enables the device to be removed from the aortic wall.

### Ventilation Means And Detection of Ventilation Completion

Furthermore, the device comprises ventilation means that allows air bubbles that may be present in the vessel, to escape to the atmosphere. The cannula 105 is a hollow tube and the puncture needle is a hollow tube concentrically arranged inside the cannula 105. The puncture needle is sharpened at the end meant to penetrate the blood vessel. A blood-air mixture present in the blood vessel is easily conveyed by these hollow structures to a receptacle or the like where the mixture can be visually observed, and the observer would have the opportunity to conclude whether all air is vented out or some air is still present in the blood vessel.

The lower end 103 of the anchoring element is fixed to an outer tube, cannula 105, which is affixed under a small collection vessel or receptacle 170. When the device with its anchoring element is attached to the inside wall of the vessel, the anchoring element 101 provides means for air ventilation such as openings on the side of the anchoring element which are formed when the tube is deployed in its expanded shape. During open heart surgery, air bubbles have a tendency to collect underneath the upper wall of the blood vessel and will therefore move along this upper wall until they reach the evacuation opening(s) on the side of the anchoring element 101. The air bubbles will escape through the openings of the anchoring element, rise through the tube of the cannula 105, exit near the cannulas upper end, and collect in the receptacle 170, which receptacle may be attached to the first sleeve 119. The opening may be arranged as a cut at the side of the cannula and the upper end opening at the end of the cannula sealed. Air is thus ventilated passively from the vessel through the anchoring element and collects as an air/blood mixture in the receptacle 170 wherein it can be visually inspected. When air is ventilated air bubbles will form in the air/blood mixture. When the flow of air bubbles is terminated and is changed into a flow of only blood it can be concluded that all of the air present in the vessel has been evacuated. A condition of “ventilation completed” is thus detected.

### Suction

As an alternative or a complement, the air can be removed by the aid of suction, wherein a suction tube is air tight connected to the receptacle 170 by means of a 90 degree bend element 410 adapted to fit snugly in the receptacle, which 90 degree bend element 410 and suction tube (not shown) enables the air to be actively removed, see FIGS. 4a and 4b.

### Infusion

The device could also be used to infuse cold cardioplegic solution aimed for a temporary cardiac arrest. The caregiver may then connect a cardioplegic infusion set to the 90 degree bend element 410. Then, during a certain period of time, the liquid will be infused through the cannula and in to the aortic root and further pass into the coronary arteries.

### Grooved Cap For Needle Control

The device may be provided with a grooved cap 201 and mechanics for convenient operation of the puncture needle used to punctuate the blood vessel. The grooved cap is a cylindrical cap with longitudinal grooves arranged at its inner surface. The grooved cap 201 is arranged over the second end of the puncture needle 140 to confer mechanical movement to the puncture needle in a controlled fashion as will be explainer further below. The grooved cap 201 is attached to a first cap arm 205 and a second cap arm 210 arranged to confer a movement from the first outer arm 131 and the second outer arm 132 to the grooved cap 201. When the outer arms 131, 132 are pressed together this entails that the distal portions 133, 134 of the outer arms 131, 132 also are pressed together, and the first cap arm 205, and the second cap arm 210 presses the grooved cap 201 downwards. This arrangement will free the index finger of the person operating the device, as compared to pressing the puncture needle more directly via a knob, as described above in connection with FIGS. 1a-1d. A weak spring, not shown, is arranged concentrically about the puncture needle, and abutting the grooved cap at one end, and the second sleeve portion 120 at the other end, for safely retracting the puncture needle 140 when the outer arms are released.

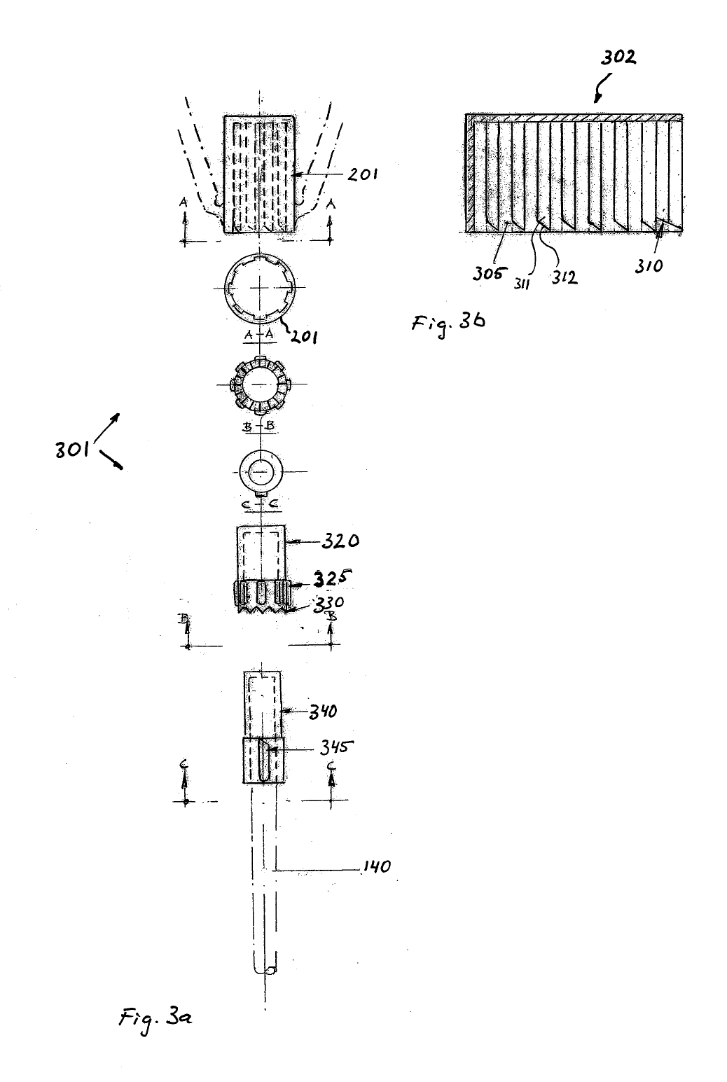

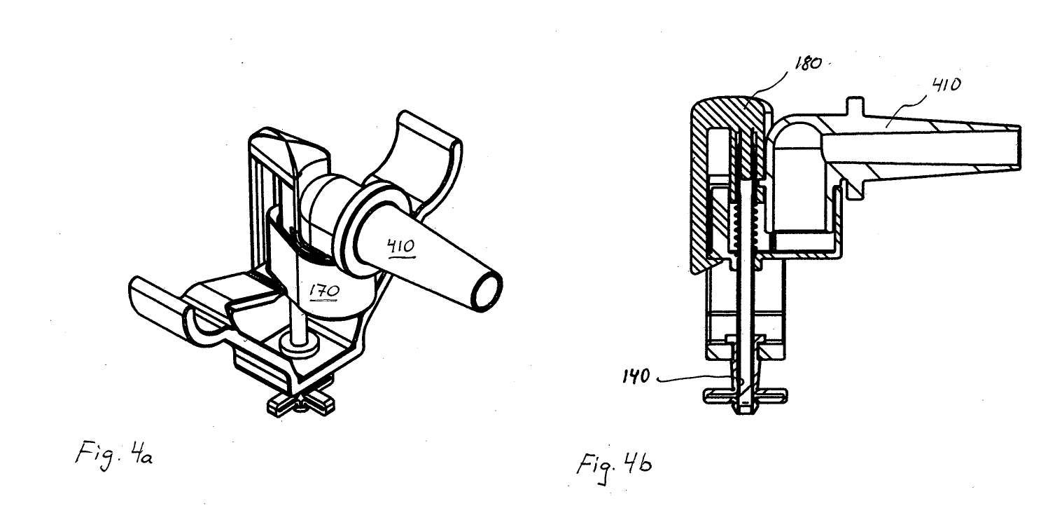

The device may further comprises parts of a protrusion-retraction mechanism 301 that translates this downward movement to a protrusion or refraction movement of the puncture needle 140 as will be explained below. FIG. 3a shows the various parts, some also in cross section, of such a protrusion-refraction mechanism 301 for the puncture needle 140 of the cannula device. Further parts comprise an axial cap 320 provided with anti rotation bars 325 and pin teeth 330, a rotor 340 provided with a rotor pin 345 on it outer surface, extending from lower end and a part of the height upwards. Upper edge of rotor pin 345 is slanted. The grooved cap 201 is cylindrical in shape and is provided with guide grooves 305 extending longitudinally on its inner cylindrical surface. Typically eight grooves are arranged evenly distributed over the inner circumference of the inner surface of the grooved cap 201. However, one of the eight groves is a blinded groove 310, that is, the groove is filled approximately 50% of it depth. To further illustrate this, FIG. 3b shows a fold-open view of the grooved cap 201, with one (ordinary) groove 305 provided with reference 305, and the blinded groove referenced with 310.

The rotor 340 is fixedly attached to the puncture needle 140, which needle 140 in turn is concentrically arranged inside the cannula 105. The axial cap is arranged to concentrically house part of the rotor 340 in such a way that the pin teeth 330, arranged at a lower end of the axial cap 320, will be able to cooperate with the upper, slanted surface of the rotor pin 345. The pin teeth is a zigzag surface arranged such that when the axial cap is pressed downwards, the pin teeth 330 cooperates with the rotor pin to rotate the rotor 340 typically one third of the distance between two grooves 305, measured centre to centre. When the pressure on the outer arms 131, 132 is released the rotor and the attached puncture needle is pressed upwards by the cylindrical spring (not shown) and the rotor pin cooperates with a slanted surface 312 of a guide bar 311 of the grooved cap 201 to rotate the rotor a further two thirds of the distance between two grooves (measured centre to centre). The rotor pin 345 subsequently slides into a groove 305 and the puncture needle 140 retracts into the cannula 105, except for that time when the blinded groove ends up in front of the rotor pin 345, at this time the puncture needle stays protruded. The anti-rotation bars 325 are arranged to prevent the axial cap 320 from rotating by engaging the grooves 305 of the grooved cap. The anti-rotation bars are arranged to have a height of a fraction of the depth of the grooves 305 to be able to move freely in the longitudinal direction in spite of the blinded groove 310. As an alternative, although less attractive from a manufacturing and assembly point of view, one of the anti-rotation bars, the one corresponding to the blinded groove, could be arranged having no height at all, leaving a no-bar surface at a location corresponding to the blinded groove.

The gist is that the puncture needle will only become deployed one time, e.g. the first time the arms are depressed, because of the blinded groove, and will not be deployed until the arms have been depressed further eight times, to engage the blinded groove again.

As an alternative, a non-grooved cap may be arranged instead of the grooved cap and is arranged to engage the puncture needle directly, without any rotation means such as the axial cap 320, or the rotor 340. The non-grooved cap is however, connected to the outer arms 131, 132 with cap arms 205, 210 in the same way as the non grooved cap 201. This simpler arrangement will also free the index finger of the person operating the device, as compared to pressing the puncture needle more directly via a knob, as described above in connection with FIGS. 1a-1d. However it entails that the puncture needle is deployed every time the outer arms 131, 132 is pressed together. A weak cylindrical spring, not shown, may preferably be arranged concentrically about the puncture needle, and abutting the grooved cap at one end, and abutting the second sleeve portion 120 at the other end, for safely retracting the puncture needle 140 when the outer arms 131, 132 are released.

1. A vascular access device suitable for evacuating air from a blood vessel having a vessel wall comprising
a cannula for insertion into the blood vessel;
characterised by
an anchoring organ having a first end and a second end, the first end of the anchoring organ being fixed to a first end of the cannula, and the anchoring organ being adapted to alter its shape between an out-spread shape and a streamlined shape upon a movement being applied to it;
a linkage comprising:
a first sleeve portion being slideable along the cannula;
a second sleeve portion being fixedly attached to the cannula;
a first (right) outer arm and a second (left) outer arm, symmetrically arranged relatively to the cannula, and opposite each other, the right outer arm and the left outer arm being mechanically connected to the first sleeve portion and being pressable by a persons fingers, wherein the first sleeve portion of the linkage is arranged closer to the first end portion of the cannula, and a first end of a left inner arm is connected to the second sleeve portion, a second end of the inner left arm is connected to an outer left arm at a point a first distance from a second end of the outer left arm, a first end of the outer left arm is connected to the first sleeve portion, and the inner left arm is made shorter than the outer left arm.

2. The device of claim 1 wherein, upon pressing, the anchoring organ assumes the streamlined shape, enabling the anchoring organ to be inserted through an opening in the wall of the blood vessel, and wherein the resilience of the hinges of the linkage are adapted to allow the anchoring organ to resume the first, out-spread shape, when the pressure is removed, thereby clamping the blood vessel wall between an outspread portion of the anchoring organ and the first sleeve portion of the linkage.

3. The device of claim 1 wherein the cannula is arranged to transfer a fluid from the blood vessel to a receptacle, the receptacle being fixed to the second sleeve portion, the fluid being a blood-air mixture, and wherein the air portion is supposed to decrease down to zero percent during an air evacuation procedure, and wherein the receptacle is arranged to enable a viewer to observe air bubbles forming in the outpouring fluid.

4. The device according to claim 1 wherein the device is provided with connecting means to connect the receptacle to an active suction device.

5. The device according to claim 1 wherein the device is provided with connecting means to connect the receptacle to an infusion aggregate.

6. The device according to claim 1 wherein the device is provided with a puncture needle concentrically arranged inside the cannula and attached to a knob.

7. The device according to claim 6 wherein the knob is arranged to be able to press with a persons index finger when holding the device between the persons thumb and middle finger of the same hand.

8. The device according to claim 7 wherein the knob is spring loaded to retract the puncture needle to a position wherein the tip is protected by the cannula, when the knob is not pressed.

9. The device according to claim 1 wherein the device is provided with a puncture needle concentrically arranged inside the cannula and being connected to a cap, the cap being connected to the outer arms via cap arms such that a movement pressing together the outer arms is transferred to a deployment of the puncture needle.

10. The device of claim 9 further comprising a mechanism having a multitude of grooves and a rotor for deploying and retracting the puncture needle in a cyclic pattern wherein the deployed state is less frequent than the retracted state upon repeated pressing of the outer arms, because one of the grooves is a blinded groove.
